(19) United States
(12) Patent Application Publication
(10) Pub. No.: US 2002/0106384 A1
(43) Pub. Date: Aug. 8, 2002

(54) Cosmetic compositions and methods for lightening the skin

(76) Inventors: Kelly Hua Zhang, Piscataway, NJ (US); Prasanna Satpute, Sparta, NJ (US); Koichi Iwata, Ridgefield Park, NJ (US); Michael Tallman, Park Ridge, NJ (US)

Correspondence Address: UNILEVER, PATENT DEPARTMENT, 45 RIVER ROAD, EDGEWATER, NJ, 07020, US

(21) Appl. No.: 09/731,964

(22) Filed: Dec. 7, 2000

Related U.S. Application Data
(60) Provisional application No. 60/170,139, filed on Dec. 10, 1999.

(51) Int. Cl. A61K 31/56; A01N 45/00; A61K 7/48; C07C 49/743

(52) U.S. Cl. 424/401; 424/62; 424/748; 514/169; 514/885; 514/886; 514/887

(57) ABSTRACT

Cosmetic compositions and methods for lightening the skin using a low molecular weight fraction of gugulipid in combination with an alpha-hydroxy acid, or niacinamide, or L-phenylalanine.

This application claims the benefit of U.S. provisional application No. 60/170,139 filed Dec. 10, 1999.

### FIELD OF THE INVENTION

The present invention is directed to cosmetic compositions and methods for lightening the skin using a gugulipid fraction in combination with an alpha-hydroxy acid, or niacinamide, or L-phenylalanine.

### BACKGROUND OF THE INVENTION

Skin lightening is desired in two instances: some consumers wish to lighten the natural color of their skin and some wish to eliminate or minimize skin blotchiness or mottling (age spots).

The color of human skin is determined by melanin, a biopolymer pigment manufactured by special dendritic cells known as melanocytes residing mostly between the basal cells of the epidermis. The biochemical process responsible for the production of melanin is caused by the action of an enzyme called tyrosinase which triggers a cascade of biosynthesis. The exact mechanism of melanin biosynthesis is a complex process. Although primary regulation of melanin production is via genetic controls, environmental factors may also play an important role in synthesis. Exposure to sunlight or other UV radiation can stimulate the melanocytes to produce more melanin, hence the so-called “tanning” reaction. Melanin production can also increase in response to hormone fluctuations associated with aging, child bearing or the use of birth-control pills.

Normal pigmentation of the skin surface is uniform. Localized, excessive pigmentation can occur and such colorization is collectively referred to as hyperpigmentation. Hyperpigmentation encompasses a wide array of afflictions all of which are accompanied by increased melanin production. Hyperpigmentation of the human skin may include skin blemishes or disorders including freckles, senile lentigo, liver spots, melasma, brown or age spots, sunburn pigmentation, post-inflammatory hyperpigmentation due to abrasion, bums, wounds, insect bites, dermatitis, and other similar small, fixed pigmented lesions.

From a cosmetic standpoint, it may often be desirable to decolorize what is considered normally pigmented skin to increase “fairness” or to blend hyperpigmented regions into that of the surrounding normal skin.

Niacinamide, L-phenyl-alanine, and alpha-hydroxy acids are among the known skin lightening agents. It has been found, as part of the present invention, that when these molecules are combined with gugulipid, a synergistic increase in skin lightening is observed. Such combinations are advantageous, since gugulipid has other skin beneficial properties, in addition to skin lightening.

Gugulipid is obtained from guggal. Guggal is obtained from a gum/resin of the plant Commiphora mukul or Commiphora wightii. Guggal contains a complex mixture of terpenes, sterols, esters and higher alcohols. The ethyl acetate extract of the resin is an oily resinous material known as “gugulipid” or “guggal lipid.”

Bombardelli et al. (U.S. Pat. No. 5,273,747) discloses the anti-inflammatory activity of gugulipid and a guggulsterone-enriched fraction thereof and their use in the treatment of benign prostatic hypertrophy and in the treatment of acne. The guggulsterone-enriched fraction described by Bombardelli was obtained with ethyl acetate and did not separate compounds by molecular weight. By contrast, in the present invention a low molecular weight fraction of gugulipid is employed.

Bissett et al. (U.S. Pat. Nos. 4,847,071 and 4,847,069) and Piazza et al. (U.S. Pat. No. 5,521,223) disclose photoprotective and anti-wrinkle compositions containing guggal as a natural anti-inflammatory. McCook et al., U.S. Pat. No. 5,690,948, discloses cosmetic antisebum and antioxidant compositions containing gugulipid or an alcoholic fraction thereof. McCook et al. do not teach the use of a low molecular weight fraction. Since gugulipid is dark-brown color and has a tar-like consistency at room temperature, it is difficult to work with and it is difficult to manufacture commercially attractive compositions with gugulipid. The alcoholic fraction has the same shortcomings as gugulipid and, in addition, its yield is relatively low.

WO 97/10196 (Parfums Christian Dior) discloses two active molecules isolated from guggal through alcoholic extraction. The actives are said to be useful in cosmetics as anti-wrinkle agents. WO 98/30199 (Unilever) discloses cosmetic compositions containing a low molecular fraction of gugulipid, as an anti-oxidant and anti-sebum agents. According to WO '199, anti-acne agents may be included, among which salicylic acid is mentioned.

None of the art cited above discloses that a combination of a low molecular weight fraction of gugulipid increases the skin lightening effect of certain skin lightening molecules. Indeed, such finding is surprising because the prior art teaches that gugulipid acts as a pigmenting agent. See WO 96/03033. In light of this teaching, it is surprising and counter-intuitive that gugulipid enhances the skin lightening activity of certain skin lightening agents.

### SUMMARY OF THE INVENTION

The present invention includes a cosmetic skin care composition comprising:

(a) a low molecular weight fraction of gugulipid;

(b) a compound selected from the group consisting of niacinamide, an alpha-hydroxy acid, L-phenylalanine, and mixtures thereof; and

(c) a cosmetically acceptable vehicle.

The following detailed description and the examples illustrate some of the effects of the inventive compositions. The invention and the claims, however, are not limited thereto.

### DETAILED DESCRIPTION OF THE INVENTION

Except in the operating and comparative examples, or where otherwise explicitly indicated, all numbers in this description indicating amounts of material or conditions of reaction and/or use are to be understood as modified by the word “about.” All amounts are by weight of the final composition, unless otherwise specified.

The term “lightening the skin” as used herein means lightening the skin natural color and/or removing or minimizing hyperpigmentation.

For the avoidance of doubt the word “comprising” is intended to mean including but not necessarily consisting of or composed of. In other words the listed steps or options need not be exhaustive.

The inventive compositions include a low molecular weight fraction of gugulipid. The inventive compositions may employ from 0.0001 to 10 wt. %, preferably from 0.001 to 3 wt. %, and most preferably from 0.01% to 2 wt. % of the low molecular weight fraction of gugulipid.

The low molecular weight fraction is obtained by dispersing or dissolving gugulipid in a polar solvent, such as alcohol (e.g., methanol) and then separating by ultrafiltration to obtain a fraction of 1,000 Da or less, preferably 800 Da or less and optimally of 500 Da or less. The solvent is then evaporated under nitrogen by e.g. gentle heat/steam bath.

Gugulipid may be obtained from the following suppliers:

C. Mukul extract: Indena (Seattle, Wash.)

Pt. Cosmetique Java, Bogar (Campo R&D, Singapore)(C. wightii extract also available).

The inventive compositions include a co-active ingredient selected from the group consisting of niacinamide, an alpha-hydroxy acid, L-phenylalanine, and mixtures thereof. The preferred co-active ingredient is an alpha-hydroxy acid, most preferably DL-lactic acid, because it was shown to have the highest efficacy as compared to other compounds tested in the screening system (B16 cell assay). DL-lactic acid showed strong inhibitory effects on melanin production in a dose-dependent manner.

The co-active ingredient is employed in an amount of from 0.001 to 20 wt %, preferably from 0.01 to 15 wt %.

The skin care composition employed in the invention also includes a cosmetically acceptable vehicle or a carrier which is inert, usually an ingredient present in the highest amounts, and functioning to deliver active or performance ingredients.

Vehicles other than water can include liquid or solid emollients, solvents, humectants, thickeners and powders. An especially preferred nonaqueous carrier is a polydimethyl siloxane and/or a polydimethyl phenyl siloxane. Silicones of this invention may be those with viscosities ranging anywhere from about 10 to 10,000,000 centistokes at 25° C. Especially desirable are mixtures of low and high viscosity silicones. These silicones are available from the General Electric Company under trademarks Vicasil, SE and SF and from the Dow Coming Company under the 200 and 550 Series. Amounts of silicone which can be utilized in the compositions of this invention range anywhere from 5 to 95%, preferably from 25 to 90% by weight of the composition. The amount of vehicle may range from about 2 to about 99 wt %, preferably from about 50 to about 99%, most preferably from about 80 to 99%, by weight of the total composition.

According to the present invention, the vehicle is preferably at least 60 wt % water, by weight of the vehicle. The inventive compositions are preferably oil-water emulsions.

Optional Skin Benefit Materials and Cosmetic Adjuncts

A preferred optional ingredient for the inclusion in the inventive compositions is sunscreen. Sunscreens include those materials commonly employed to block ultraviolet light. Illustrative compounds are titanium dioxide, the derivatives of PABA, cinnamate and salicylate. For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the emulsions can vary depending upon the degree of protection desired from the sun's UV radiation.

Another category of functional ingredients within the cosmetic compositions of the present invention are thickeners. A thickener will usually be present in amounts anywhere from 0.1 to 20% by weight, preferably from about 0.5% to 10% by weight of the composition. Exemplary thickeners are cross-linked polyacrylate materials available under the trademark Carbopol from the B. F. Goodrich Company. Gums may be employed such as xanthan, carrageenan, gelatin, karaya, pectin and locust beans gum. Under certain circumstances the thickening function may be accomplished by a material also serving as a silicone or emollient. For instance, silicone gums in excess of 10 centistokes and esters such as glycerol stearate have dual functionality.

Powders may be incorporated into the cosmetic composition of the invention. These powders include chalk, talc, Fullers earth, kaolin, starch, smectite clays, chemically modified magnesium aluminum silicate, organically modified montmorillonite clay, hydrated aluminum silicate, fumed silica, aluminum starch octenyl succinate and mixtures thereof.

Other adjunct minor components may also be incorporated into the cosmetic compositions. These ingredients may include coloring agents, opacifiers and perfumes. Amounts of these other component materials may range anywhere from 0.001% up to 20% by weight of the composition.

Use of the Composition

The composition according to the invention is intended primarily as a product for topical application to human skin, especially as an agent for lightening the skin.

In use, a small quantity of the composition, for example from 1 to 100 ml, is applied to exposed areas of the skin, from a suitable container or applicator and, if necessary, it is then spread over and/or rubbed into the skin using the hand or fingers or a suitable device.

According to the present inventive method, the skin lightening by the active ingredient is attained by topical application of corn seed extract.

The following specific examples further illustrate the invention, but the invention is not limited thereto.

### EXAMPLE 1

The ability to inhibit melanin production was assessed using B16 cells, a mouse melanoma cell line.

Methodology

B16 F1 Melanoma Cell Based Assay for Melanogenesis

B16-F1 mouse melanoma cells (obtained from American Type Culture Collection, MD, USA) were maintained in 75 cm² culture flasks in RPMI 1640 medium supplemented with L-glutamine (4 mM) and 10% fetal bovine serum (FBS) at 37° C. in a water-saturated, 5% CO₂ in air atmosphere. Sub-confluent B16 cells were seeded in 96 well microtiter plates at a density of 5×10³ cells/well in 200 μl of DMEM containing 10% FBS and 1% penicillin/streptomycin without phenol red at 37° C. under 5% CO₂. After 24 hours, the media was replaced with fresh DMEM media containing various compounds at designated concentrations. Cells were incubated for 72 hours at which time melanin was visible in the control well (dark color) where no testing compounds were added.

Melanin Content in Media

The amount of melanin produced for each well was quantified by assessing melanin released into the culture media. The pigment containing media from each well was transferred to a clean 96 well plate and the melanin content was quantified by reading the absorbance at 530 nm. Melanin content in each well was calculated as a percent of the control where cells were maintained in DMEM medium or solvent control.

Melanin content in the media was expressed as percent of control, therefore the lower the value is, the more inhibition resulted from the tested compounds.

Cell Viability Assay

Cell viability was analyzed by neutral red dye absorption to establish whether pigmentation inhibition effects identified were caused as a result of cytotoxicity or cell proliferation as opposed to inhibition of melanogenesis. For example, a good inhibitor for melanin synthesis (melanin: 20% of control, viability: 100% of control) can be distinguished from a bad inhibitor (melanin: 20% of control, viability: 20% of control).

Cell viability was assessed by neutral red dye uptake. After the removal of media, 200 μl of pre-warmed neutral red solution (25 μg/ml media) was added to each well and incubated for 3 hours under conditions as for cell maintenance. Cells were washed 2× with PBS. The dye was extracted by adding 100 μl of solvent (50:49:1 of H₂O:ethanol:acetic acid) and then gently shaken at room temperature for 20 minutes. The dye was quantified by reading the absorbance at 530 nm.

Preparation of the Low Molecular Weight Fraction of Gugulipid (Hereinafter “Gugul Select”):

The gugulipid ethyl acetate extract as supplied by Indena (Milan, Italy) was dissolved to a final concentration of 6.5% (w/v) gugulipid using the HPLC grade methanol. After 10-15 minutes, a clear, medium brown solution resulted. Upon standing, a fine talc-like, white precipitate was observed to make a thin coating at the bottom of the container. Gentle swirling of the solution caused the precipitate to slightly opacify the solution, but this would again clear if the solution was allowed to stand agitated for a few minutes.

The resulting solution was vigorously stirred during sample removal so that a representative aliquot of the solution and precipitate could be removed. A total volume of 50 ml was removed and delivered to solvent holding chamber of an Amicon Ultrafiltration cell model 8400 equipped with a Diaflo YCO5 (500 Da cut-off) 76 mm ultrafiltration membrane (both from Millipore Corp.). Sample elution was performed by pressurizing the solvent holding chamber with nitrogen gas, and thereby forcing the sample through the ultrafiltration membrane and out the effluent line. The maximal pressure used to filter the solution did not exceed 55 psi. Once the sample had been decreased to 1-2 ml, the system was vented and 25 ml of clean methanol was introduced to the solvent holding chamber. This was then passed through the filter to the same point and repeated again with 15 ml of methanol.

All effluent was collected in the same container (approximately 90 ml) and represented the low molecular weight fraction. The retentate material was collected by extensively rinsing of the filter and holding chamber with methanol. This material was then collected together and represented the high molecular weight fraction material.

The containers containing both fractions were placed on a Pierce Reacti-therm III heating module equipped with a Reacti-vap III nitrogen drying assembly. Under a constant nitrogen stream of 2 psi, the heat was slowly increased over 20 min. to a maximum setting of 4-5 (setting range 1 [low] to 10 [high]). These conditions were maintained until the samples reached complete dryness. After allowing the containers to cool to room temperature, samples were re-dissolved in small amounts of methanol and quantitatively transferred to pre-tarred test tubes for mass determination.

84% of the gugulipid was recovered in a low molecular weight fraction (“gugul select”) which was tested at 0.0001% in ethanol for its effects on melanin synthesis.

Results

### EXAMPLE 1

Combination of 0.0001% of gugul select with niacinamide at 2, 5, 10, and 15 mM concentration showed significant (n=4, p<0.05) inhibition in melanin synthesis in B16 F1 cells. The combination was significantly better than either niacinamide or gugul select alone. There were no cytotoxic effects from the treatments as indicated by a cell viability of ≧91.5% of control.

| | | Melanin | | |
|---|---|---|---|---|
| | Melanin | (% Control) | P value | P value |
| Niacinamide | (% Control) | With | vs. | vs. |
| (mM) | Alone | gugul select | Gugul select | Niacinamide |
| 0 | 100 ± 20 | 105.4 ± 21.4 | 1.00 | — |
| 2 | 94.8 ± 19 | 57.3 ± 18.7 | <0.05 | <0.05 |
| 5 | 83.9 ± 16.7 | 54.9 ± 16.4 | <0.05 | <0.05 |
| 10 | 66.1 ± 13 | 37.6 ± 12.9 | <0.05 | <0.05 |
| 15 | 50.3 ± 10.2 | 24.8 ± 7.1 | <0.05 | <0.05 |

### EXAMPLE 2

Combination of 0.0001% of gugul select with DL-lactic acid at 2, 5, 10, and 15 mM concentration showed significant (n=4, p<0.05) inhibition in melanin synthesis in B16 F1 cells. The combination was significantly better than either DL-lactic acid or gugul select alone. There were no cytotoxic effects from the treatments as indicated by a cell viability of ≧91.6% of control.

| | | Melanin | | P value |
|---|---|---|---|---|
| DL-lactic | Melanin | (% Control) | P value | vs. |
| acid | (% Control) | With | vs. | DL-lactic |
| (mM) | Alone | gugul select | Gugul select | acid |
| 0 | 100 ± 20 | 105.4 ± 21.4 | 1.00 | — |
| 2 | 103.8 ± 20.5 | 54.7 ± 13.1 | <0.05 | <0.05 |
| 5 | 98 ± 19.3 | 19.9 ± 6.3 | <0.05 | <0.05 |
| 10 | 79 ± 16.8 | 11 ± 3 | <0.05 | <0.05 |
| 15 | 33.5 ± 7.9 | 5.5 ± 1.3 | <0.05 | <0.05 |

### EXAMPLE 3

Combination of 0.0001% of gugul select with L-phenylalanine at 2, 5, 10, and 15 mM concentration showed significant (n=4, p<0.05) inhibition in melanin synthesis in B16 F1 cells. The combination was significantly better than either L-phenylalanine or gugul select alone. There were no cytotoxic effects from the treatments as indicated by a cell viability of ≧103.7% of control.

| | | Melanin | | |
|---|---|---|---|---|
| L-phenyl- | Melanin | (% Control) | P value | P value |
| alanine | (% Control) | With | vs. | vs. |
| (mM) | Alone | gugul select | Gugul select | Phenylalanine |
| 0 | 100 ± 20 | 105.4 ± 21.4 | 1.00 | — |
| 2 | 84.9 ± 16.9 | 44.2 ± 27.3 | <0.05 | <0.05 |
| 5 | 48.3 ± 10 | 9 ± 3.7 | <0.05 | <0.05 |
| 10 | 10 ± 2.7 | 6 ± 1.5 | <0.05 | <0.05 |
| 15 | 49.7 ± 9.9 | 5.9 ± 2 | <0.05 | <0.05 |

It should be understood that the specific forms of the invention herein illustrated and described are intended to be representative only. Changes, including but not limited to those suggested in this specification, may be made in the illustrated embodiments without departing from the clear teachings of the disclosure. Accordingly, reference should be made to the following appended claims in determining the full scope of the invention.

What is claimed is:

1. A cosmetic skin care composition comprising:
(a) a low molecular weight fraction of gugulipid;
(b) a co-active compound selected from the group consisting of niacinamide, an alpha-hydroxy acid, L-phenylalanine, and mixtures thereof; and
(c) a cosmetically acceptable vehicle.

2. The composition of claim 1 comprising from about 0.001 to about 10 wt. % of the gugulipid.

3. The composition of claim 1 comprising from about 0.001 to about 20 wt. % of the co-active compound.

4. The composition of claim 1 wherein the low molecular weight fraction of gugulipid has the molecular weight below about 1,000 Da.

5. A cosmetic method of lightening the skin or minimizing hyperpigmentation, the method comprising applying to the skin the composition of claim 1.
